(19) United States
(12) Patent Application Publication
(10) Pub. No.: US 2007/0191757 A1
(43) Pub. Date: Aug. 16, 2007

(54) Method and Device for Sublingual Drug Delivery Using Iontophoresis

(75) Inventors: Solomon S. Steiner, Mount Kisco, NY (US); Robert Feldstein, Yonkers, NY (US); Roderike Pohl, Sherman, CT (US); David Rhodes, Storrs, CT (US); Erik Steiner, New York, NY (US)

Correspondence Address: PATREA L. PABST;PABST PATENT GROUP LLP, 400 COLONY SQUARE, SUITE 1200, 1201 PEACHTREE STREET, ATLANTA, GA, 30361, US

(73) Assignee: Biodel Inc.

(21) Appl. No.: 11/675,430

(22) Filed: Feb. 15, 2007

Related U.S. Application Data
(60) Provisional application No. 60/773,817, filed on Feb. 16, 2006.

(51) Int. Cl. A61N 1/30

(52) U.S. Cl. 604/20

(57) ABSTRACT

Methods, devices and kits for sublingual drug delivery using iontophoresis are described herein. An active agent can be administered sublingually by placing a solid oral dosage form containing the active agent in the sublingual region of a patient and applying iontophoresis for a suitable period of time. Preferably up to 4 mA of current are applied to the sublingual region. Different time ranges can be used to administer iontophoresis; preferably iontophoresis is administered for up to 2 minutes at a time. Any suitable device for administering iontophoresis to the sublingual region may be used. The preferred device is a hand-held device that contains a handle, two electrodes, one of which is located on the handle and the other of which is attached to the end of the handle, and a connection to a power source. Optionally, the device contains a timer, which can be used turn off the current at a preset time. The device can be used to administer an active agent by iontophoresis to the sublingual region of a patient, by attaching the second electrode of the device to a solid oral dosage form containing the active agent to be administered. A kit contains the device for administering iontophoresis and one or more solid oral dosage forms, preferably in the form of one or more tabs or wafers. The tabs or wafers may be completely dissolvable or edible, or may contain a non-edible and non-dissolvable component. In a preferred embodiment, the solid oral dosage form contains insulin or an analog thereof and one or more excipients, preferably EDTA and citric acid.

### CROSS-REFERENCE TO RELATED APPLICATION

This application claims priority to U.S. Ser. No. 60/773,817, entitled “Method and Device for Sublingual Drug Delivery using Iontophoresis” to Solomon S. Steiner, Robert Feldstein, Roderike Pohl, David Rhodes, and Erik Steiner, filed Feb. 16, 2006.

### FIELD OF THE INVENTION

The present invention is directed to the field of methods and devices for sublingual drug delivery using iontophoresis.

### BACKGROUND OF THE INVENTION

Absorption of any molecule, such as a nutrient or pharmaceutical agent, from the oral cavity and more specifically from the lingual and sublingual regions involves a different route than absorption of molecules through the gastrointestinal tract. Molecules absorbed from the stomach and small intestines of the gastrointestinal tract are collected in the hepatic portal drainage system and go directly to the liver where they are exposed to a variety of enzymes which degrade many therapeutics and nutrients, including peptides, proteins and oligonucleotides. In contrast, molecules absorbed from the oral, lingual and sublingual regions go directly into the systemic circulation, bypassing the hepatic portal system and avoiding initial exposure to the enzymes in the gastrointestinal tract. The oral cavity is a gentler environment for many molecules, especially peptides and proteins, compared to the harsh acidic and peptide-lysing environment of the stomach and intestines. Furthermore, the underside and base of the tongue, as well as the base of the oral cavity beneath the tongue, are highly variegated and vascularized, containing capillaries close to the surface, which presents a considerable surface area to allow rapid absorption of a desired drug or nutrient.

For the above reasons, the oral cavity and more specifically, the lingual and sublingual regions of the oral cavity, appear to be an ideal site for the delivery of many therapeutic and nutritional molecules, including peptides and proteins. However, while a number of drugs have been successfully delivered by this route, there remain a number of problems with this mode of delivery.

One problem with using the oral cavity for drug delivery is due to a patient's automatic swallowing response. A liquid placed in the oral cavity in amounts greater than 200 microliters (μL) will usually elicit a swallowing response, removing the drug to be delivered from the oral cavity and subjecting it to the harsh conditions of the stomach. As a result, most of the drug delivered to the buccal cavity is no longer available for absorption from the oral, lingual and sublingual regions. This reduces the bioavailability of the drug.

Attempts have been made to overcome this problem by affixing the drug to a film or other adhesive that adheres to the sublingual space, trapping the drug next to the absorptive surface. However this approach presents many limitations. For example, the base of the tongue is irregularly shaped and variegated and as a result standardized patches do not readily adhere to it. Further, an individual who has a foreign substance located under the tongue normally automatically reacts by moving the position of the foreign substance and swallowing it.

WO 2005/072803 to Biodel, Inc. discloses drug delivery devices that aerosolize a dry powder formulation of a therapeutic agent or nutrient and dispense the formulation so that it forms a fine coating in the sublingual region of the oral cavity. However, other types of devices can be used to administer a drug to the sublingual region.

Therefore it is an object of the invention to provide a device for administering drugs to the oral cavity, particularly the sublingual region.

It is a further object of the invention to provide an improved method for administering drugs through the sublingual region of the oral cavity.

### BRIEF SUMMARY OF THE INVENTION

Methods, devices and kits for sublingual drug delivery using iontophoresis are described herein. An active agent can be administered sublingually by placing a solid oral dosage form containing the active agent in the sublingual region of a patient and applying iontophoresis for a suitable period of time. Preferably up to 4 mA of current are applied to the sublingual region. Different time ranges can be used to administer iontophoresis; preferably iontophoresis is administered for up to 2 minutes at a time. Any suitable device for administering iontophoresis to the sublingual region may be used. The preferred device is a hand-held device that contains a handle, two electrodes, one of which is located on the handle and the other of which is attached to the end of the handle, and a connection to a power source. Optionally, the device contains a timer, which can be used turn off the current at a preset time. The device can be used to administer an active agent by iontophoresis to the sublingual region of a patient, by attaching the second electrode of the device to a solid oral dosage form containing the active agent to be administered. A kit contains the device for administering iontophoresis and one or more solid oral dosage forms, preferably in the form of one or more tabs or wafers. The tabs or wafers may be completely dissolvable or edible, or may contain a non-edible and non-dissolvable component. In a preferred embodiment, the solid oral dosage form contains insulin or an analog thereof and one or more excipients, preferably ethylenediaminetetracetic acid (EDTA) and citric acid.

### BRIEF DESCRIPTION OF THE DRAWINGS

FIG. 1A is a side-view of the hand-held device attached to solid oral dosage form. FIG. 1B is a top view of the hand-held device attached to solid oral dosage form.

FIG. 2 is an illustration showing use of the hand-held device attached to solid oral dosage form. FIG. 2 shows a cross-section of a patient's mouth in which the solid oral dosage form has been placed and the patient's hand holding the handle of the device.

FIG. 3 is an illustration showing the experimental set up for the Example. FIG. 3 shows the use of a modified IOMED® (available from Iomed, Inc. Corp., Salt Lake City, Utah) device attached to solid oral dosage form for sublingual drug delivery in a mini-pig.

FIG. 4 is an illustration of the adhesive portion of the IOMED® device.

FIG. 5 is a graph of time (minutes) versus blood concentration of insulin (μU/ml) in mini-pigs for three different formulations, sublingual administration of insulin with excipient (“Viavolt”) (solid line), subcutaneously administered insulin (broken line, long dashes), and sublingual administration of a low pH insulin formulation (broken line, short dashes). The horizontal, solid line in the lower left-hand corner of the graph indicates the time period during which iontophoresis was applied.

### DETAILED DESCRIPTION OF THE INVENTION

I. Device

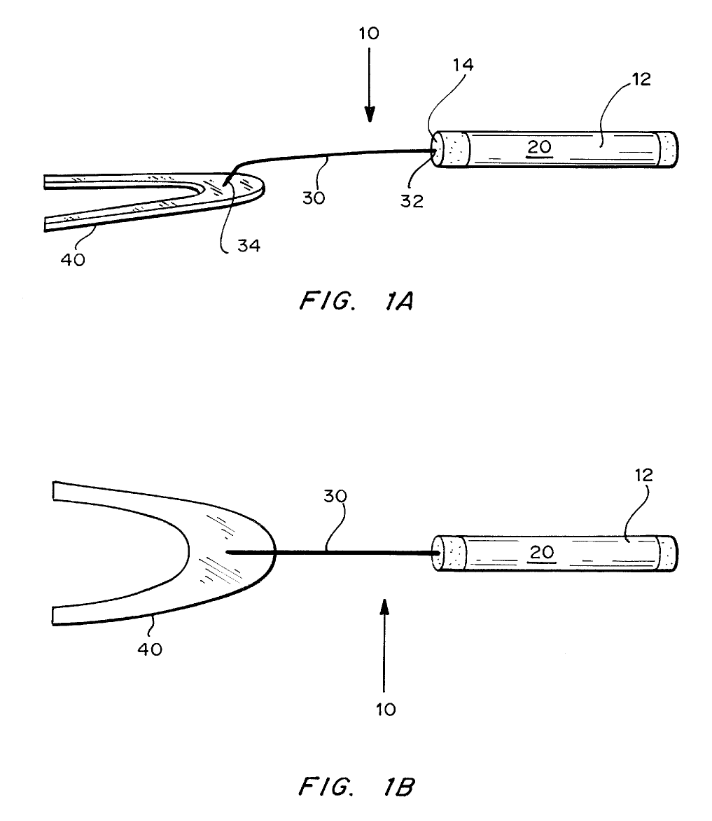

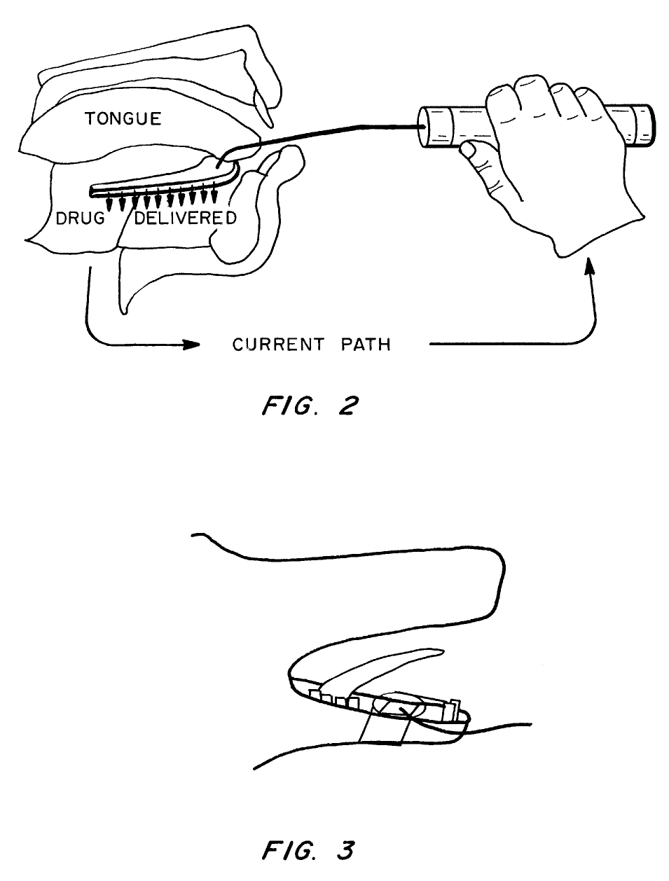

An exemplary hand-held device (10) is illustrated in FIGS. 1A and 1B. The device typically contains a handle (12), two electrodes (20 and 30), and a connection to a power source (not shown in Figure). The device is preferably designed to deliver up to 4 mA of current. Optionally, the device contains a timer (not shown in Figure), which can be used turn off the current at a preset time.

The device can be used to administer a drug by iontophoresis to the sublingual region of a patient. Iontophoresis involves the application of an electrical current, preferably DC or pulsating DC, at a current density of greater than zero up to about 160 mA/cm². Typically, a constant current is applied since resistance changes over time, usually driven by a voltage between zero and four volts. Application of electric current enhances transdermal transport. For example, application of an electric field provides an additional driving force for the transport of charged molecules across the skin and second, ionic motion due to application of electric fields may induce convective flows across the skin, referred to as electrosmosis. This mechanism is believed to play a dominant role in transdermal transport of polarizable molecules during iontophoresis.

A. Handle

The handle (12) is designed to be held by a patient. Preferably the handle is light-weight. Although the handle may have any shape, the handle is preferably in the shape of a cylinder. Preferably the handle is up to 5 inches long and up to 8 inches in circumference. The surface of the handle may be made of any conductive material, such as steel, iron, copper, gold, silver, platinum, palladium, and aluminum since the handle contains the first electrode (20). Preferably the surface of the handle is a smooth surface.

B. First Electrode

The first electrode (20) is designed to be held by a patient, and is located on the surface of the handle.

C. Connection to Power Source

Preferably the handle contains a connection to a power source (not shown in Figure). The connection may be in the form of a holder for a battery, or a wire. In the most preferred embodiment, the connection is in the form of a holder for a battery. Optionally, the device contains the power source, preferably in the form of one or more batteries and control circuitry.

D. Second Electrode

The second electrode (30) is designed to connect to a solid oral dosage form. One end (32) of the second electrode (30) is attached to one end of the handle (14). The end (34) of the electrode that is distal to the handle (12) is designed to connect to the solid oral dosage form (40). Connection may occur by insertion of the electrode into a space on the solid oral dosage form or by attachment of the electrode to the solid oral dosage form, such as through the use of a connector.

II. Kit

A typical kit, in its fully assembled form, is illustrated in FIGS. 1A and 1B. The kit contains the device and one or more solid oral dosage forms, preferably in the form of one or more tabs or wafers. Optionally the kit also contains a power source, preferably in the form of one or more batteries and control circuitry.

1. Solid Oral Dosage Forms

The solid oral dosage form contains the active agent to be delivered. The solid oral dosage form is preferably in the form of a tab or wafer. The solid oral dosage form may have any suitable shape, such as arc-shaped, oval, circle, and square. Preferably the solid oral dosage form is arc-shaped, to allow for the dosage form to be placed in the mouth beneath and surrounding the user's tongue (40, in FIGS. 1A and 1B). The solid oral dosage form contains a space designed for the placement of an electrode, and/or a connector, which can attach to an electrode. The internal resistance of the solid oral dosage form should be lower, and is preferably substantially lower, than the resistance of the target tissue. The solid oral dosage form could be in the form of or contain a conductive mesh, conductive film, or conductive comb structure.

The dosage form may be formed of components that dissolve in the mouth or are edible, or may contain a non-edible matrix that does not dissolve in the mouth. In one embodiment, the connector is non-edible and does not dissolve in the mouth.

A. Edible and Dissolvable Solid Oral Dosage Forms

In one embodiment, the solid oral dosage form is a tab or wafer having an essentially homogenous composition. In another embodiment, the tab or wafer is a bilayer or trilayer tab or wafer. The bilayer tab or wafer preferably contains one layer that dissolves more slowly that the other layer, preferably the layer that dissolves more slowly contains a space or connector for attaching the second electrode to this layer. The trilayer tab or wafer preferably contains one layer that dissolves more slowly that each other two layers, preferably this layer is the middle layer. In the preferred embodiment, the trilayer tab or wafer contains a first layer containing the drug to be delivered, a second layer that dissolves more slowly than the other two layers, and a third layer containing the drug to be delivered. Optionally, the second layer is edible. Preferably, the solid oral dosage form contains a space designed for the placement of an electrode. If the solid oral dosage form is a bilayer or trilayer tab or wafer, the space for insertion of the electrode may be located in one or more of the layers. Preferably the space is located in a layer that dissolves more slowly than the other layer(s) and is more conductive than the outer surface of the solid oral dosage form, which interfaces with the epithelial tissue. Such a design for the solid oral dosage form provides uniform current density throughout the dosage form.

B. Solid Oral Dosage Forms Containing at Least one Non-Edible, Non-Dissolvable Component

In yet another embodiment, the solid oral dosage form contains a conductive matrix that does not dissolve in the mouth and is not-edible. Optionally, the matrix may be reusable by a patient. Suitable materials for the matrix include gold, silver, platinum, palladium, aluminum, steel, iron, and copper. The solid oral dosage form may be in the form of a bilayer or trilayer tab or wafer. The matrix contains a connector, which is designed to attach to the second electrode. In one embodiment, the connector is non-edible and does not dissolve in the mouth.

C. Active Agents

The solid oral dosage form may be used to deliver a wide range of active agents, including peptides, proteins, nucleotide molecules (RNA sequences, DNA sequences), sugars, polysaccharides, and small organic molecules. Preferably, the active agent is at least slightly soluble in aqueous medium (i.e. 10,000 parts of aqueous solvent per solute), and more preferably is highly soluble in aqueous medium. Preferably the active agent is highly potent, so that only a small amount (e.g. in the microgram range) is needed to provide a therapeutic effect. Suitable peptides include but are not limited to insulin and derivatives of insulin, such as Lispro; C-peptide; glucagon-like peptide 1 (GLP 1) and all active fragments thereof; human amylin and synthetic forms of amylin, such as pramlinitide; parathyroid hormone (PTH) and active fragments thereof (e.g. PTH₁₋₃₄); calcitonin; human growth hormone (HGH); crythropoietin (EPO); macrophage-colony stimulating factor (M-CSF); granulocyte-macrophage-colony stimulating factor (GM-CSF); and interleukins. In the preferred embodiment the active agent is insulin. Suitable small molecules include nitroglycerin, sumatriptan, narcotics (e.g. fenatnyl, codeine, propoxyphene, hydrocodone, and oxycodone), benzodiazepines (e.g. Alprazolam, Clobazam, Clonazepam, Diazepam Flunitrazepam, Lorazepam, Nitrazepam, Oxazepam, Oxazepam, Temazepam, and Triazolam), phenothiazines (Chlorpromazine, Fluphenazine, Mesoridazine, Methotrimeprazine, Pericyazine, Perphenazine, Prochlorperazine, Thioproperazine, Thioridazine, and Trifluoperazine), and selective serotonin reuptake inhibitors (SSRIs) (e.g. sertraline, fluvoxamine, fluoxetine, citalopram, and paroxetine).

In the preferred embodiment, the active agent is insulin or an analog or derivative thereof. The insulin can be recombinant or purified. In the preferred embodiment, the insulin is human insulin. Recombinant human insulin is available from a number of sources. Preferably the insulin is administered in the presence of one or more excipients, such as a chelator and/or solubilizing agent, that dissolve rapidly in aqueous media. Preferably solubilizers such as acids and metal chelators are included in the dosage form.

The dosages of the active agents depend on their bioavailability and the disease or disorder to be treated, as well as the individual pateint. Insulin is generally included in a dosage range of 12 to 2000 IU per human dose. Thus if the insulin has a bioavailability 5-25%, the actual systemie dose delivered to an individual ranges from 3 to 100 IU. For insulin with only 2.5% bioavailability, an oral dose of 4,000 IU will deliver a 100 IU systemically available dose. For insulin with a much greater bioavailability, such as a 50% bioavailability, the delivery of a 3 IU systemically available dose requires an oral dose of 6 IU.

D. Excipients

The solid oral dosage form typically includes one ore more excipients. Preferably, at least one of the excipients is selected to mask any charges on the active agent. This facilitates the transmembrane transport for the active agent and thereby increases both the onset of action and bioavailability for the active agent. The excipients are also selected to form compositions that dissolve rapidly in aqueous medium. Optional pharmaceutically acceptable excipients present in the solid oral dosage form include, but are not limited to diluents, binders, lubricants, disintegrants, colorants, stabilizers, and surfactants.

Solubilizing Agents

In the preferred embodiment, one or more solubilizing agents are included with the active agent to promote rapid dissolution in aqueous media. Suitable solubilizing agents include wetting agents such as polysorbates and poloxamers, non-ionic and ionic surfactants, food acids and bases (e.g. sodium bicarbonate), and alcohols, and buffer salts for pH control. suitable acids include acetic acid, ascorbic acid, citric acid, amino acids and hydrochloric acid. For example, if the active agent is insulin, a preferred solubilizing agent is citric acid.

Chelators

In the preferred embodiment, a metal chelator is mixed with the active agent or in a coating surrounding the active agent. The chelator may be ionic or non-ionic. Suitable chelators include ethylenediaminetetraacetic acid (EDTA), citric acid, dimercaprol (BAL), penicillamine, alginic acid, chlorella, cilantro, alpha lipoic acid, dimercaptosuccinic acid (DMSA), dimercaptopropane sulfonate (DMPS), and oxalic acid. In the preferred embodiment, the chelator is EDTA. The chelator hydrogen bonds with the active agent, thereby masking the charge of the active agent and facilitating transmembrane transport of the active agent. For example, when the active agent is insulin, in addition to charge masking, it is believed that the chelator pulls the zinc away from the insulin, thereby favoring the monomeric form of the insulin over the hexameric form and facilitating absorption of the insulin by the tissues surrounding the site of administration (e.g. mucosa, or fatty tissue). Optionally, the chelator and solubilizing agent are the same compound.

Ions may be part of the active agent, added to the stabilizing agent, mixed with the chelator, and/or included in the coating. Representative ions include zinc, calcium, iron, manganese, magnesium, aluminum, cobalt, copper, or any di-valent metal or transitional metal ion. Zn⁺² has a stronger binding preference for EDTA than Ca⁺².

Diluents and Fillers

Diluents, also referred to herein as fillers, are typically necessary to increase the bulk of a solid dosage form so that a practical size is provided for compression of tablets or formation of beads and granules. Suitable fillers include, but are not limited to, dicalcium phosphate dihydrate, calcium sulfate, lactose, sucrose, mannitol, sorbitol, cellulose, microcrystalline cellulose, powdered cellulose, kaolin, sodium chloride, dry starch, hydrolyzed starches, pregelatinized starch, silicone dioxide, titanium oxide, magnesium aluminum silicate, calcium carbonate, compressible sugar, sugar spheres, powdered (confectioner's ) sugar, dextrates, dextrin, dextrose, dibasic calcium phosphate dehydrate, glyceryl palmitostearate, magnesium carbonate, magnesium oxide, maltodextrin, polymethacrylates, potassium chloride, talc, and tribasic calcium phosphate.

Binders

Binders are used to impart cohesive qualities to a solid oral dosage form, and thus ensure that a tab or wafer remains intact after its formation. Suitable binder materials include, but are not limited to, starch, pregelatinized starch, gelatin, sugars (including sucrose, glucose, dextrose, lactose and sorbitol), dextrin, maltodextrin, zein, polyethylene glycol, waxes, natural and synthetic gums such as acacia, guar gum, tragacanth, alginate, sodium alginate, celluloses, including hydroxypropylmethylcellulose, carboxymethylcellulose sodium, hydroxypropylcellulose, hydroxylethylcellulose, ethylcellulose, methyl cellulose, and veegum, hydrogenated vegetable oil, Type I, magnesium alumninum silicate, and synthetic polymers such as acrylic acid and methacrylic acid copolymers, carbomer, methacrylic acid copolymers, methyl methacrylate copolymers, aminoalkyl methacrylate copolymers, polyacrylic acid/polymethacrylic acid, and polyvinylpyrrolidone.

Lubricants

Examples of suitable lubricants include, but are not limited to, magnesium stearate, calcium stearate, stearic acid, glyceryl behenate, glyceryl monostearate, glyceryl palmitostearate, hydrogenated castor oil, hydrogenated vegetable oil, type I, sodium benzoate, sodium lauryl sulfate, sodium stearyl fumarate, polyethylene glycol, talc, zinc stearate, and mineral oil and light mineral oil.

Disintegrants

Disintegrants are used to facilitate dosage form disintegration or “breakup” after administration, and generally include, but are not limited to, starch, sodium starch glycolate, sodium carboxymethyl starch, methylcellulose, calcium carboxymethylcellulose, sodium carboxymethylcellulose, hydroxypropyl cellulose, microcrylstalline cellulose, colloidal silicon dioxide, croscarmellose sodium, pregelatinized starch, clays, cellulose, powdered cellulose, pregelatinized starch, sodium starch glycolate, sodium aginate, alginic acid, guar gum, magnesium aluminum silicate, polacrilin potassium, and cross linked polymers, such as cross-linked PVP, crospovidone (POLYPLASDONE® XL from GAF Chemical Corp).

Stabilizers

Stabilizers are used to inhibit or retard drug decomposition reactions which include, by way of example, oxidative reactions. A number of stabilizers may be used. Suitable stabilizers include polysaccharides, such as cellulose and cellulose derivatives, and simple alcohols, such as glyccrol; bacteriostatic agents such as phenol, m-cresol and methylparaben; isotonic agents, such as sodium chloride, glycerol, and glucose; lecithins, such as example natural letchins (e.g. eg yolk lecithin or soya bean lecithin) and synthetic or semisynthetic lecithins (e.g. dimyristoylphosphatidylcholine, dipalmitoylphosphatidylchloline or distearoyl-phosphatidylcholine; phosphatidic acids; prosphatidylethanolamines; phosphatidylserines such as distearoyl-phosphatidylserine, dipalmitoylphosphatidylserine and diarachidoylphospahtidylserine; phosphatidylglycerols; phosphatidylinositols; cardiolipins; sphingomyelins; and synthetic detergents, such as diosctanoylphosphatidyl choline and polyethylenepolypropylene glycol). Other suitable stablizers include acacia, albumin, alginic acid, bentonite, carboxymethylcellulose calcium, carboxymethylcellulose sodium, cyclodextrins, glyceryl monostearate, hydroxypropyl cellulose, hydroxypropyl methylcellulose, magnesium aluminum silicate, propylene glycol, propylene glycol alginate, sodium alginate, white wax, xanthan gum, and yello wax. In the preferred embodiment, the agent is insulin and the stabilizer may be a comination of one or more polysaccharides and glycerol, bacteriostatic agents, isotonic agents, lecithins, or synthetic detergents.

Surfactants

Surfactants may be anionic, cationic, amphoteric or nonionic surface active agents. Suitable anionic surfactants include, but are not limited to, those containing carboxylate, sulfonate and sulfate ions. Examples of anionic surfactants include sodium, potassium, ammonium of long chain alkyl sulfonates and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)-sulfosuccinate; and alkyl sulfates such as sodium lauryl sulfate. Cationic surfactants include, but are not limited to, quaternary ammonium compounds such as benzalkonium chloride, benzethonium chloride, cetrimonium bromide, stearyl dimethylbenzyl ammonium chloride, polyoxyethylene and coconut amine. Examples of nonionic surfactants include ethylene glycol monostearate, propylene glycol myristate, glyceryl monostearate, glyceryl stearate, polyglyceryl-4-oleate, sorbitan acylate, sucrose acrylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, Poloxamer® 401, stearoyl monoisopropanolamide, and polyoxyethylene hydrogenated tallow amide. Examples of amphoteric surfactants include sodium N-dodecyl-β-alanine, sodium N-lauryl-β-iminodipropionate, myristoamphoacetate, lauryl betaine and lauryl sulfobetaine.

If desired, the solid oral dosage form may also contain minor amount of nontoxic auxiliary substances such as dyes, sweeteners, coloring and flavoring agents, pH buffering agents, or preservatives.

E. Polymers

Blending or copolymerization sufficient to provide a certain amount of hydrophilic character can be useful to improve wettability of the materials in the solid oral dosage form. For example, about 5% to about 20% of monomers may be hydrophilic monomers. Hydrophilic polymers such as hydroxylpropylcellulose (HPC), hydroxpropylmethylcellulose (HPMC), carboxymethylcellulose (CMC) are commonly used for this purpose. Also suitable are hydrophobic polymers such as polyesters and polyimides. It is known to those skilled in the art that these polymers may be blended with polyanhydrides to achieve compositions with different drug release profiles and mechanical strengths. Preferably, the polymers are bioerodable, with preferred molecular weights ranging from 1000 to 15,000 Da, and most preferably 2000 to 5000 Da.

F. Preferred Insulin Solid Oral Dosage Form

In a preferred embodiment, the solid oral dosage form is a tab or a wafer containing insulin. Preferred insulin dosages included 75, 150, 300 and 600IU doses. In the most preferred embodiment, the insulin tab or wafer also contains EDTA and citric acid. Preferably, for every 1 mg of insulin (1 mg insulin is equivalent to about 27IU insulin), the dosage form contains between 0.1 mg and 10 mg EDTA and between 0.1 mg and 10 mg citric acid. In the most preferred embodiment, the dosage form contains insulin, EDTA and citric acid in a mass ratio of 1:2:2 (mass insulin:mass EDTA:mass citric acid). In the preferred embodiment the wafer or tab is in the shape of an arc. Preferably the wafer or tab is lyophilized and stored in frozen state under dry conditions until use.

III. Methods of Using Iontophoresis to Improve Drug Delivery

An active agent can be administered sublingually by placing a dosage form containing the active agent in the sublingual region of a patient and applying iontophoresis for a suitable period of time. Preferably up to 4 mA of current are applied to the sublingual region. Different time ranges can be used to administer iontrophoresis. Preferably the current is applied for up to 2 minutes at a time.

The pateint's mouth should not be dry when administering the drug. Typically, a few minutes prior to placement of the solid oral dosage form in the pateint's mouth, the patient should take a small drink of water to ensure that hir/her mouth is sufficiently moist.

If the tab or wafer is stored ina frozen state, the temperature of the tab or wafer is preferably allowed to come to room temperature prior to placement in the patient's mouth.

The tab or wafer is typically connected to the second probe, either via insertion of the second probe into the tab or wafer, or through the use of a suitable connector.

Then the tab is placed in the patient's mouth, under the tongue. If the tab or wafer is in the shape of an arc, the tab or wafer is placed in the mouth as illustrated in FIGS. 1A and 1B, with the narrow ends towards the back of the mouth and on either side of the tongue. Then the patient places his/her tongue over the tab or wafer, closes his/her mouth. The patient's mouth preferably remains closed throughout the application of iontophoretic procedure. Once the patient has closed his/her mouth, the current can be turned on.

If a device as described above is used to deliver the current, the current will pass from the handle through the tab or wafer, and make a complete circuit through the body back to the hand held device. The current will help to administer the dose through the sublingual epithelium.

Preferably, the device contains a timer, and the timer is set to shut off the device at a preset time limit, preferably after three minutes of turning on the device.

Once the current is turned off, the second electrode may be removed from the mouth.

If the tab or wafer is completely dissolvable, the tab or wafer should be left in place under the tongue until fully dissolved. The patient should be instructed to try not to swallow the dose during this time period.

If the tab or wafer is not completely dissolvable, the tab or wafer should be left in place under the tongue for a period of time sufficient to allow for transfer of the drug from the solid oral dosage form into the sublingual region. Typically, the tab or wafer should remain in the sublingual region for time ranging from 0.5 minutes to 20 minutes, preferably for a time period ranging from 2 minutes to 5 minutes, after turning off the current.

Although the method of using iontophoresis for improved sublingual drug delivery is described herein with reference to the hand-held device and kit described above, any suitable source of iontrophoresis can be used.

As shown in the Example, below, an IOMED® device (available from Iomed, Inc. Corp., Salt Lake City, Utah), which is a commercially available adhesive patch for administering iontophoresis transdermally, can be modified to create a device suitable for sublingual drug delivery. If a modified IOMED® device is used, the device does not contain a handle, and a first electrode is placed on the body, such as through the use of an adhesive patch, or held in the patient's hand, and the second electrode is placed in an absorbent pad. If the drug formulation is in the form of a liquid, it can be added to the absorbent pad using a syringe. Then the drug-loaded absorbent pad, which is attached to the second electrode, is placed in the patient's mouth, under the patient's tongue (see FIG. 3). The device is turned on to deliver the iontrophoresis. Preferably the device contains a timer, which is set to automatically turn off the device at a pre-set time.

The present invention will be further understood by reference to the following non-limiting example.

### EXAMPLE

### Example 1

### Mini-Pig Study of Sublingual Drug Delivery using Iontophoresis

Three mini pigs were used in this study. At least two pre-dose blood glucose levels and 1 ml blood samples were taken from each mini pig. A 1 ml blood sample was taken from a catheter in a cephalic vein and was transferred to an EDTA-containing collection tube on ice and processed per standard operating procedure. The glucose levels were determined using a Therasense blood glucose meter.

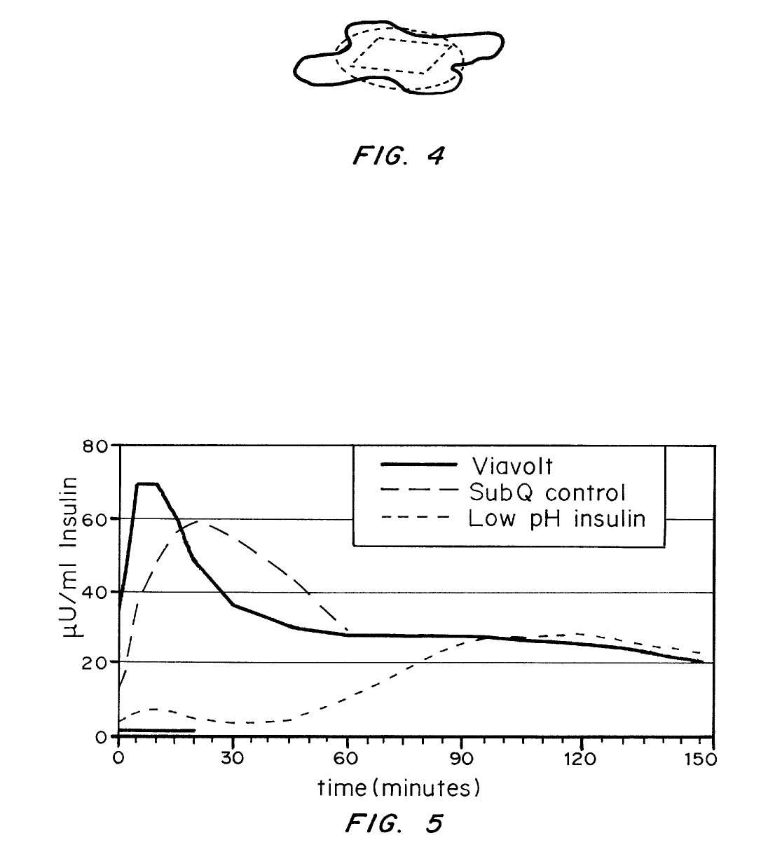

The fore-shoulder of the pig was shaved, thoroughly cleaned, and completely dried. A rectangular (black adhesive side) IOMED® dosing patch having a diameter of 2.5 cm was stuck to this region so as to provide good electrical contact with the skin. The edges of the IOMED® dosing patch were trimmed so as to remove the excess adhesive tabs but leave a circle of adhesive around the perimeter of the absorbent pad (see FIG. 4). The circle was approximately 6 cm in diameter. The black clip of the IOMED® device was attached to the contact in the center of the patch. The red clip of the device was attached to the contact in the center of the other patch (see FIG. 3). A syringe was used to add 2.5 ml of a solution containing 1 mg insulin/ml, 2 mg EDTA/ml and 2 mg citric acid/ml (referred to herein as “Viaject”) to an IOMED® pad. The Viaject dose contained 25 IU of insulin/, with 62.5 IU of insulin administered per patch. Two control doses were used. The first control dose was a solution of insulin at pH 4.0 in the absence of excipients (25 IU/ml×2.5 ml) added by syringe to an IOMED® pad, with 62.5 IU of insulin per patch administered as described above. The second control dose was administered as a subcutaneous injection, at a dose of 0.1 IU of insulin/kg.

As illustrated in FIG. 3, the pad was placed in the mouth of the mini pig, positioned so as to place the pad as close to the base of the tongue as possible. Graze was used to hold the patch in place with the electrical lead free to connect to the dosing device.

The device was set for 20 minutes at 4 mA and activated. This was “time zero” and the time was recorded on the data sheet. At the intervals specified on the data sheet, blood samples were taken, and the time and blood glucose level were recorded.

Results

Blood insulin data from the study are shown in FIG. 5. The dashed line is the control data from a subcutaneous injection and is similar to the results obtained with other experiments using this animal model and to clinical results with human volunteers using subcutaneously administered Viaject.

The solid line is the data from iontrophoretic delivery of Viaject. The dotted line is data from iontophoretic delivery of insulin at the same pH as that in Viaject (same molecular charge), but without the excipients. The gray bar is the time during which the delivery device was activated.

Conclusion

The delivery of insulin by iontophoresis was significantly faster than delivery by subcutaneous injection. It should be kept in mind that the comparator in this experiment was Viject, which has been shown to appear in the blood more rapidly than conventional human insulin for injection. The excipients present in Viaject, appear to serve as an important part of the delivery system since the control experiment with acidified insulin alone was not rapidly delivered.

Unless defined otherwise, all technical and scientific terms used herein have the same meanings as commonly understood by one of skill in the art to which the disclosed invention belongs. Publications cited herein and the material for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

We claim:

1. A hand-held device for drug delivery comprising two electrodes and a connection to a power source, wherein one electrode is suitable for insertion into a patient's mouth.

2. The device of claim 1, further comprising a handle, wherein the first electrode is located on the surface of the handle and wherein the second electrode is connected to one end of the handle.

3. The device of claim 1, further comprising a timer.

4. The device of claim 2, wherein the connection to a power source is located inside the handle.

5. A kit comprising a solid oral dosage form and the hand-held device comprising two electrodes and a connection to a power source, wherein one electrode is suitable for insertion into a patient's mouth, wherein the solid oral dosage form comprises a space or connector suitable for the placement of the second electrode.

6. The kit of claim 5, wherein the hand-held device further comprises a handle, wherein the first electrode is located on the surface of the handle and wherein the second electrode is connected to one end of the handle.

7. The kit of claim 5, wherein the hand-held device further comprises a time.

8. The kit of claim 5, wherein the solid oral dosage form is a wafer to tab.

9. The kit of claim 8, wherein the wafer or tab is in the shape of selected from the group consisting of arc-shaped, oval, circle, and square.

10. The kit of claim 9, wherein the wafer or tab is in the shape of an arc with a hollow center.

11. The kit of claim 8, wherein the wafer or tab is a bilayer or trilayer wafer of tab.

12. The kit of claim 5, wherein the solid oral dosage form is selected from the group consisting of solid oral dosage forms containing at least one non-edible, non-dissolvable component and dissolvable or edible solid oral dosage forms.

13. The kit of claim 5, wherein the solid oral dosage form comprises an active agent and one or more excipients.

14. The kit of claim 13, wherein the active agent is selected from the group consisting of peptides, proteins, nucleotide molecules, sugars, polysaccharides, and small organic molecules.

15. The kit of claim 14, wherein the active agent is insulin or an analog thereof.

16. The kit of claim 15, wherein the one or more excipients comprise EDTA and citric acid.

17. A method for sublingual drug delivery comprising
providing a kit to a patient in need thereof, wherein the kit comprises a solid oral dosage form and the hand-held device comprising two electrodes and a connection to a power source, wherein one electrode is suitable for insertion into a patient's mouth, wherein the solid oral dosage form comprises a space or connector suitable for the placement of the second electrode,
attaching the solid oral dosage form to the second electrode,
placing the solid oral dosage form in the sublingual region of the patient's mouth, and
turning on the device for a suitable period of time to apply iontophoresis through the solid oral dosage form to the sublingual region of the patient's mouth.

18. The method of claim 17, wherein the hand-held device further comprises a handle, wherein the first electrode is located on the surface of the handle and wherein the second electrode is connected to one end of the handle.

19. The method of claim 17, wherein the hand-held device further comprises a timer, and wherein the method further comprises setting the timer for a suitable period of time prior to turning on the device.

20. The method of claim 17, further comprising turning off the device, waiting for a time period ranging from 0.5 minutes to 20 minutes, and then chewing or swallowing the remainder of the solid oral dosage form or removing the non-edible or non-dissolvable portion of the solid oral dosage form from the patient's mouth.
